SEQUENCE LISTING (1 sequence)

SEQ ID NO: 1 - protein, 14 aa, Homo sapiens
  misc_feature (7)..(7): Xaa=Dpa= (3,(2,4-dinitrophenyl)-L-2,3- diaminopropionique amide)
PLAQAVXAARSSSR

(19) United States
(12) Patent Application Publication
(10) Pub. No.: US 2010/0168089 A1
(43) Pub. Date: Jul. 1, 2010

(54) UTILIZATION OF TACE INHIBITORS FOR THE TREATMENT OF ACNE

(75) Inventors: JÉRÔME AUBERT, GRASSE (FR); ISABELLE CARLAVAN, GRASSE (FR); JOHANNES VOEGEL, CHATEAUNEUF/GRASSEE (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/640,029

(22) Filed: Dec. 17, 2009

Related U.S. Application Data
(63) Continuation of application No. PCT/FR2008/051085, filed on Jun. 18, 2008.

(30) Foreign Application Priority Data
Jun. 18, 2007 (FR) 0755819

(51) Int. Cl. A61K 31/541; A61K 31/4406; A61K 31/16; A61K 31/381; A61K 31/18; A61K 31/4402; A61K 31/426; A61K 31/47; A61K 31/5377; A61K 31/395; A61K 31/497; A61K 31/517; A61P 17/10; C12Q 1/68; C12Q 1/37

(52) U.S. Cl. 514/227.8; 514/357; 514/616; 514/445; 514/605; 514/352; 514/371; 514/314; 514/236.2; 514/183; 514/254.1; 514/266.3; 435/6; 435/23

(57) ABSTRACT

An in vitro method of screening for candidate compounds for the preventive or curative treatment of acne entails determination of the ability of a compound to inhibit the expression or the activity of TACE, and also to the administration of inhibitors of the expression or of the activity of this enzyme, in the treatment of acne.

### CROSS-REFERENCE TO PRIORITY/PCT APPLICATIONS

This application claims priority under 35 U.S.C. §119 of FR 07/55819, filed Jun. 18, 2007, and is a continuation/national phase of PCT/FR 2008/051085, filed Jun. 18, 2008 and designating the United States (published in the French language on Jan. 8, 2009 as WO 2009/004247 A2; the title and abstract were also published in English), each hereby expressly incorporated by reference in its entirety and each assigned to the assignee hereof.

### BACKGROUND OF THE INVENTION

1. Technical Field of the Invention

The present invention relates to the identification and the utilization of compounds that modulate the TACE enzyme, for the treatment of acne.

2. Description of Background and/or Related and/or Prior Art

Acne is a skin condition which results from the occlusion of the upper end and also of the internal part of the pilosebaceous canal owing to abnormal keratinocyte multiplication, and the androgenic hormone hyperactivity which often appears during puberty, which causes a considerable increase in seborrhea in the sebaceous glands. The obstruction of the pilosebaceous canal causes the formation of comedones or microcysts, accompanied by proliferation of Propionibacterium acnes bacteria and also of Pytirosporum ovale in the obstructed pilosebaceous follicles.

This condition, which is particularly common in adolescents, is accompanied by an inflammatory reaction of the skin, that may be in the form of papules or pustules generally located in the superficial dermis. In certain cases, the inflammatory reaction may reach the deep dermis, forming nodules and macrocysts.

Conventional treatments for acne use benzoyl peroxide, erythromycin, isotretinoin and various antiseptics. However, the use of antibiotics such as erythromycin today encounters the phenomena of bacterial resistance and modifications of flora.

### SUMMARY OF THE INVENTION

The present invention features utilization of inhibitors of the TACE enzyme, for preventing and/or improving acne phenomena.

Preferably, the TACE inhibitor is employed alone, i.e., without any other anti-acne active ingredient, or even without any other active ingredient.

The acne to be treated comprises all forms of acne, in particular, common acne, comedonal acne, polymorphic acne, nodulocystic acne, acne conglobata, or else secondary acne such as solar acne, acne medicamentosa or occupational acne.

### TACE

The TACE enzyme, for TNF-alpha converting enzyme, is responsible for the production of TNF-alpha. This enzyme is a member of the ADAM family of metalloproteases (Becherer et al, Handb. Exp. Pharmacol., 2000, 140:235-258). It rapidly converts proTNFα, a 26 kDa precursor protein, to the mature 17 kDa TNFα protein. The inhibition of TACE (or ADAM 17) results in the inhibition of TNFα synthesis (Nelson et al, Exp. Opin. Invest. Drugs, 1999, 8:383-392). It has, moreover, been shown that TACE inhibitors suppress TNF production and the inflammatory response in model animals suffering from collagen-induced arthritis (Newton et al, Ann Rheum. Dis., 2001, 60:iii25-iii32).

It has also been shown that P. acnes stimulates TNFα release by keratinocytes and macrophages (Gro et al, Br J. Dermatol., 2004, 150(3):421-428). An overexpression of TNFα has, moreover, been observed in acneic lesions (Kang et al, Am J. Pathol., 2005, 166(6):1691-9).

Analysis of the transcriptome of an acneic lesion at an inflammatory stage has made it possible to identify a broad panel of genes preferentially expressed in the lesion (Trivedi et al, JID. 2006, May; 126(5):1071-9). Without wishing to be bound to any particular mechanism of action, the hypothesis is now proposed that TACE inhibitors have an impact on the inflammatory phenomena observed in acne, in particular, via the modulation, by means of TNFα, of several genes involved in inflammation. Among the markers for the inflammatory acne lesion that are reported to be induced in response to exposure to TNFα, the following genes involved in the “stress response” are found:

SGK (Li X et al, J Biol Chem., 2002 Nov. 22; 277(47):45129-40);

SOD2 (Horrevoets et al, Blood 1999 May 15; 93(10):3418-31); the following gene involved in angiogenesis:

ECGF1 (Zhu et al, Oncogene 2002 Dec. 5; 21(55):8477-85); the following genes involved in cell migration and remodeling:

MMP1, MMP3 (Reunanen et al, J Biol Chem., 2002 Aug. 30; 277(35):32360-8);

T1A-2 (Banno et al, J Biol Chem., 2004 Jul. 30; 279(31):32633-42. Epub 2004 May 15);

the following genes involved in the immune response:

DEFB4 (Seo S J et al, J Dermatol Sci., 2001 November; 27(3):183-91);

IL1F9 (Kumar et al, J Biol Chem., 2000 Apr. 7; 275(14):10308-14);

CXCL2 (Banno et al, J Biol Chem., 2004 Jul. 30; 279(31):32633-42; Siwkowski et al, Mol Pharmacol, 2004 Sep. 1; 66(3):572-9);

IL-8 (Brasier et al, J Biol Chem., 1998 Feb. 6; 273(6):3551-61; Terui et al, Blood 1998 Oct. 15; 92(8):2672-80);

CCR1 (Li et al, J Biol Chem., 2002 Nov. 22; 277(47):45129-40; Sozzani et al, J Immunol., 1998 Aug. 1; 161(3):1083-6);

CD14 (Enomoto et al, J Pharmacol Exp Ther., 2003 Sep. 1; 306(3):846-54);

CD47 (Banno et al, J Biol Chem., 2004 Jul. 30; 279(31):32633-42);

FPR1 (Mandal et al, J Immunol., 2005 Nov. 1; 175(9):6085-91);

PLSCR1 (Banno et al, J Biol Chem., 2004 Jul. 30; 279(31):32633-42);

TNC (Scherberich et al, Oncogene 2005 Feb. 24; 24(9):1525-32; Banno et al, J Biol Chem., 2004 Jul. 30; 279(31):32633-42);

CYBB (Dusi et al, Eur J Immunol., 2001 Mar. 1; 31(3):929-38).

The TNFα inhibits, on the other hand, the FRZB gene involved in development (Tian et al, J Biol Chem., 2005 Apr. 29; 280(17):17435-48).

Finally, TNFα inhibits two genes involved in metabolism:

KYNU (Banno et al, J Biol Chem., 2004 Jul. 30; 279(31):32633-42),

AKR1B10 (Iwata et al, J Biol Chem., 1999 Mar. 19; 274(12):7993-8001).

Several TACE inhibitor molecules are in development, mainly for the treatment of arthritis. Many patent applications also propose TACE inhibitors for the treatment of arthritis, of cancers or of other diseases, such as U.S. Patent Publication Application No. 2004/122011 (Masferrer J L) and WO 99/42436 (American Cyanamid). Specifically, these two applications describe methods of treatment or compositions containing TACE inhibitors for the treatment of various pathological conditions, including acne.

In the context of the present invention, the term “TACE gene” or “TACE nucleic acid” signifies the gene or the nucleic acid which encodes the TACE enzyme. If the intended target is preferably the human gene or the expression product thereof, the present invention also features cells expressing a heterologous TACE, by genomic integration or transient expression of an exogenous nucleic acid encoding the enzyme.

### DETAILED DESCRIPTION OF BEST MODE AND SPECIFIC/PREFERRED EMBODIMENTS OF THE INVENTION

### Screening Methods

One aspect of the present invention is an in vitro method of screening for candidate compounds for the preventive and/or curative treatment of acne, comprising the determination of the ability of a compound to inhibit the expression or the activity of TACE or the expression of its gene or the activity of at least one of its promoters, inhibition of the expression or of the activity of TACE, or of the expression of its gene or of the activity of at least one of its promoters, indicating that the compound is of value in the preventive or curative treatment of acne.

The term “inhibition” means any reducing effect on the expression or the activity of the enzyme, on the expression of its gene or on the activity of at least one of its promoters.

The compounds tested may be of any type. They may be of natural origin or may have been produced by chemical synthesis. They may be a library of structurally defined chemical compounds, of uncharacterized compounds or substances, or of a mixture of compounds.

Various techniques may be used to test these compounds and to identify the compounds of therapeutic interest, that are modulators of the expression or of the activity of TACE.

Preferably, the screening method comprises the following steps:

a. preparing at least two biological samples or reaction mixtures;

b. bringing one of the samples or reaction mixtures into contact with one or more of the test compounds;

c. measuring the expression or the activity of the TACE enzyme, the expression of its gene or the activity of at least one of its promoters, in the biological samples or reaction mixtures;

d. selecting the compounds for which an inhibition of the expression or of the activity of the TACE enzyme, or an inhibition of the expression of its gene or an inhibition of the activity of at least one of its promoters, is measured in the sample or the mixture treated in b), compared with the untreated sample or mixture.

Throughout the present text, unless otherwise specified, the term “expression of an enzyme” means the amount of this enzyme.

The term “activity of an enzyme” means its biological activity.

The term “activity of a promoter” means the ability of this promoter to initiate transcription of the DNA sequence encoded downstream of this promoter (and there indirectly the synthesis of the corresponding protein, in this case the enzyme).

According to a first embodiment, the biological samples or reaction mixtures of step a) are cells transfected with a reporter gene functionally linked to all or part of the TACE gene promoter. The method is then carried out by bringing a compound into contact (step b)) with these biological samples or reaction mixtures, and then by determining the level of expression of said reporter gene (step c)). The selection in step d) is carried out by means of the observation of a difference in level of expression, relative to a control carried out in the absence of the compound. This difference indicates the usefulness of the compound in the preventive or curative treatment of acne.

The reporter gene may in particular, encode an enzyme which, in the presence of a given substrate, results in the formation of colored products, such as CAT (chloramphenicol acetyltransferase), GAL (beta-galactosidase) or GUS (beta-glucuronidase). It may also be the luciferase or GFP (green fluorescent protein) gene. The assaying of the protein encoded by the reporter gene, or of its activity, is carried out conventionally, by colorimetric, fluorometric or chemiluminescence techniques, inter alia.

According to a second embodiment, the biological samples or reaction mixtures of step a) are cells expressing the TACE gene encoding TACE. The screening method then comprises bringing a compound into contact (step b)) with the biological samples or reaction mixtures, and then determining the level of expression of said gene (step c)). The selection in step d) is carried out by means of the observation of a difference in level of expression of the TACE gene, relative to a control carried out in the absence of the compound. This difference indicates the usefulness of the compound in the preventive or curative treatment of acne.

The cell employed here may be of any type. It may be a cell expressing the TACE gene endogenously, for instance a liver cell, an ovarian cell, or even better still a keratinocyte or a cell of the immune system, such as THP1 cells.

It may also be a cell transformed with a heterologous nucleic acid encoding TACE, which is preferably human, or mammalian.

A large variety of host-cell systems may be used, such as, for example, Cos-7, CHO, BHK, 3T3 or HEK293 cells. It is also possible to use microorganisms such as bacteria (for example, E. coli or B. subtilis); yeasts (for example, Saccharomyces pichia); or insect cells, such as Sf9 or Sf21.

The nucleic acid may be stably or transiently transfected, by any method known to one skilled in the art, for example by means of calcium phosphate, DEAE-dextran, liposome, virus, electroporation or microinjection.

In this method, the level of expression of the TACE gene may be determined by evaluating the level of transcription of said gene, or the level of translation thereof.

The expression “level of transcription of a gene” means the amount of corresponding mRNA produced.

The expression “level of translation of a gene” means the amount of protein produced.

One skilled in the art is familiar with the techniques for quantitatively or semi-quantitatively detecting the mRNA of a gene of interest. Techniques based on hybridization of the mRNA with specific nucleotide probes are the most customary (Northern blot, RT-PCR, Rnase protection assay). It may be advantageous to also use detection labels, such as fluorescent, radioactive or enzymatic agents or other ligands (for example, avidin/biotin).

The level of translation of the gene is evaluated, for example, by methods of immunological assay type, mass spectrometry analysis (MALDI-TOF and thin-layer analysis), etc. For an immunological assay of the product of said gene, the antibodies used may be of polyclonal or monoclonal type. The production of said antibodies is the result of conventional techniques. The immunological assay may be carried out in solid phase or in homogeneous phase; in one step or two steps; in a sandwich method or in a competition method, by way of non-limiting examples. According to one preferred embodiment, the capture antibody is immobilized on a solid phase. By way of non-limiting examples of a solid phase, microplates may be employed, in particular, polystyrene microplates, or solid particles or beads, paramagnetic beads.

ELISA assays, radioimmunoassays, or any other detection technique may be used to reveal the presence of the antigen-antibody complexes formed.

The characterization of the antigen/antibody complexes, and more generally of the isolated or purified, but also recombinant, proteins (obtained in vitro and in vivo), may be carried out by mass spectrometry (proteomic) analysis.

According to a third embodiment, the biological samples or reaction mixtures of step a) are reaction mixtures comprising a TACE enzyme, or preferably a 610-amino acid N-terminal fragment of the human recombinant TACE enzyme, and a substrate for the enzyme. The screening method in this case comprises bringing a compound into contact (step b)) with these mixtures then determining the enzymatic activity (step c)). The selection in step d) is carried out by means of the observation of a decrease in activity, relative to a control carried out in the absence of the compound. Such a decrease indicates the usefulness of the compound in the preventive or curative treatment of acne.

An enzymatic activity test is described in Jin et al, Anal Biochem., 2002 Mar. 15; 302(2):269-75. This test may also be modified using a different substrate. Thus, it is possible to use, as fluorogenic substrate, the peptide MCA-PLAQAV(Dpa)RSSSR—NH₂, the amino acid sequence of which surrounds the TNFα precursor cleavage site. The cleavage site is located from the alanine and valine residues. The fluorescent donor group is MCA ((7-methoxycoumarin-4-yl)acetyl) and the quenching acceptor group is Dpa (3-(2,4-dinitrophenyl)-L-2,3-diaminopropionic amide). The activity of substrate cleavage by the human recombinant TACE enzyme is monitored by fluorescence, with excitation at 320 nm and emission at 420 nm.

Another technique in which the activity of TACE can be measured consists in measuring the production of TNFα, for example by means of an immunoassay using the HTRF (homogeneous time-resolved fluorescence) technique on keratinocytes or cultures of THP1 cells. For this, the cells are prestimulated with LPS and/or TPA, and the TNFα produced by the cells is assayed using two murine antibodies which each recognize a different epitope of human TNFα. Each antibody is conjugated either with a europium cryptate, or with an allophycocyanin XL665. This combination makes it possible to measure a FRET signal when the two antibodies each bind to one end of the TNFα produced.

The candidate TACE inhibitors can then be tested on acne models, or on patients suffering from acne. The invention thus also provides a method of screening for candidate compounds for the treatment and/or prevention of acne, said method comprising the administration of a TACE inhibitor compound to an individual with acneic or acne-prone skin, and the evaluation of the condition of the acne. The administration is preferably carried out topically. The term “topically” means application to the skin or the mucous membranes.

### Inhibitors of the Enzyme

Another aspect of the present invention is also the use of an inhibitor of the human TACE enzyme, in particular, an inhibitor obtained by means of the screening method described above, in the preparation of a medicament for use in the preventive and/or curative treatment of acne. Preferably, the medicament contains no cyclooxygenase-2(COX-2) inhibitor.

Preferably, the inhibitor of the human TACE enzyme is the only anti-acne active ingredient contained in the medicament. Even more preferably, the inhibitor of the human TACE enzyme is the only active ingredient contained in the medicament.

A method of preventive and/or curative treatment of acne is thus featured herein, said method, whether regime or regimen, comprising the administration of a therapeutically effective amount of a modulator of the human TACE enzyme to a patient requiring such a treatment.

The term “inhibitor” refers to a compound or a chemical substance which eliminates or substantially reduces the enzymatic activity of TACE. The term “substantially” signifies a reduction of at least 20%, preferably of at least 30%, more preferably of at least 50%, and more preferably of at least 70% or 90%. More particularly, the inhibitor may be a compound which interacts with, and blocks, the catalytic site of the enzyme.

A preferred inhibitor interacts with the enzyme in solution at inhibitor concentrations of less than 1 μM, preferably less than 0.1 μM, more preferably less than 0.01 μM.

The modulator compound may be an anti-TACE inhibitory antibody, preferably a monoclonal antibody. Advantageously, such an inhibitory antibody is administered in sufficient quantity to obtain a plasma concentration of approximately 0.01 μg per ml to approximately 100 μg/ml, preferably of approximately 1 μg per ml to approximately 5 μg/ml.

Other preferred inhibitors are indicated in Table 1 below:

TABLE 1
TACE inhibitors:

| Compound | |
|---|---|
| Number | Structure |
| T1 | 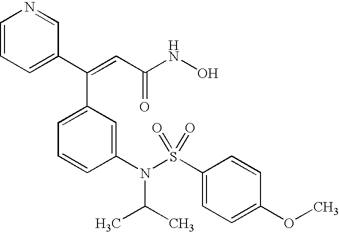 |
| T2 | 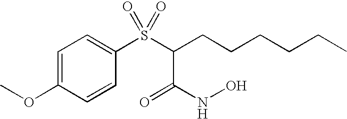 |
| T3 | 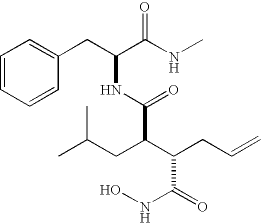 |
| T4 | 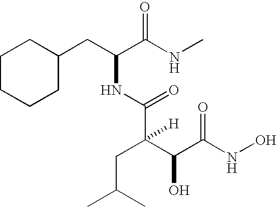 |
| T5 | 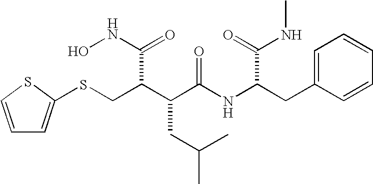 |
| T6 | 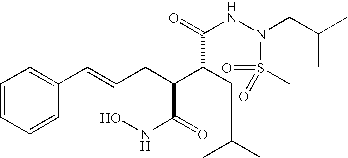 |
| T7 | 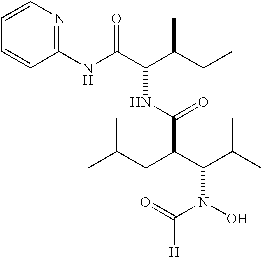 |
| T8 | 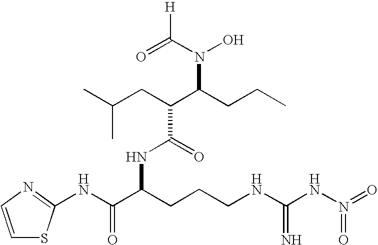 |
| T9 | 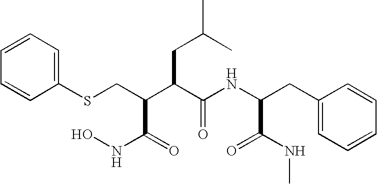 |
| T10 | 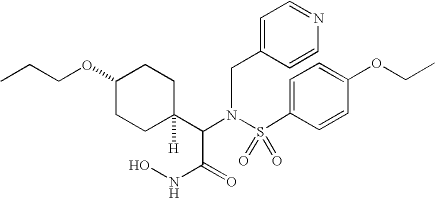 |
| T11 | 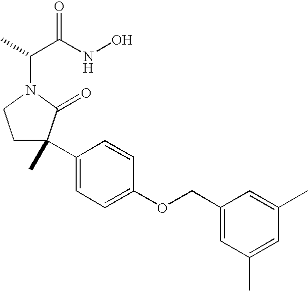 |
| T12 | 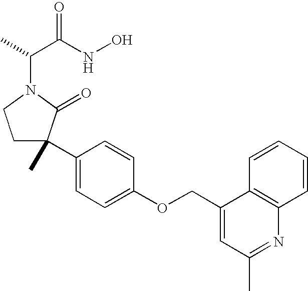 |
| T15 | 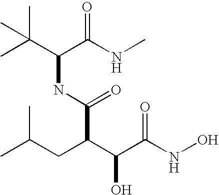 |
| T16 | 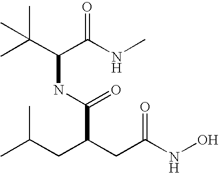 |
| T17 | 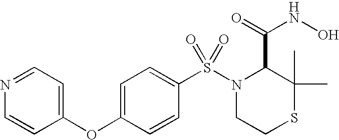 |
| T18 | 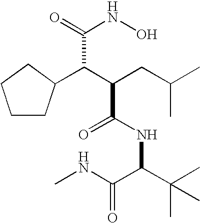 |
| T19 | 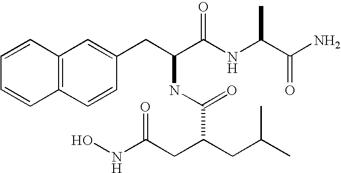 |
| T20 | 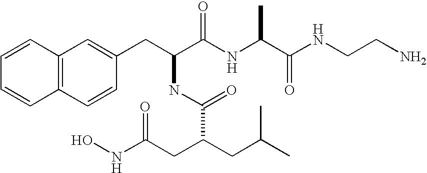 |
| T21 | 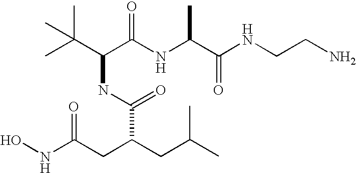 |
| T22 | 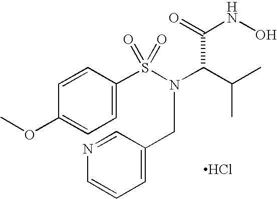 |
| T23 | 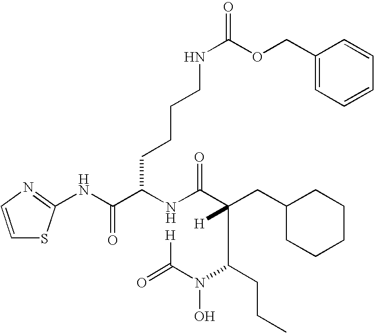 |
| T24 | 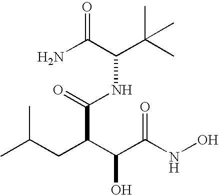 |
| T25 | 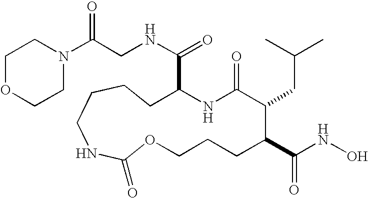 |
| T26 | 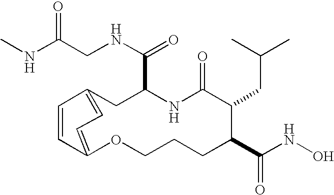 |
| T27 | 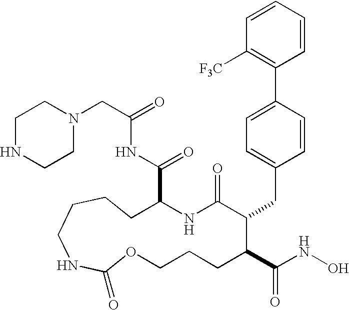 |
| T28 | 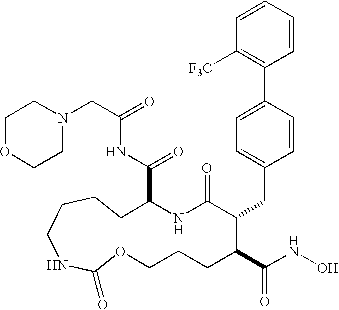 |
| T29 | 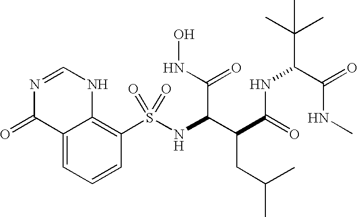 |
| T30 | 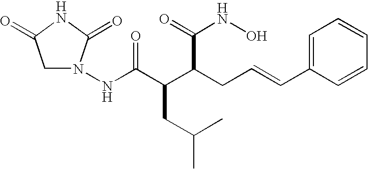 |
| T31 | 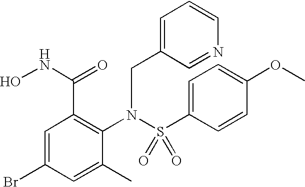 |
| T32 | 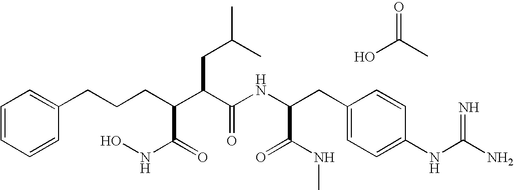 |
| T33 | 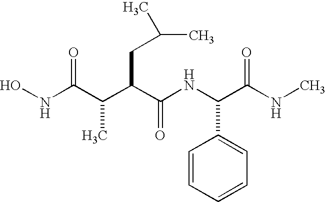 |

The names, CAS number and references of the inhibitors that are even more preferred are indicated in Table 2 below:

TABLE 2
Names of TACE inhibitors:

| No. | Name(s) | CAS | Reference |
|---|---|---|---|
| T1 | W-3646, 3-[3-[N-isopropyl-N-(4- | | Abstracts of Papers, |
| | methoxyphenylsulphonyl)amino]-phenyl]- | | 222nd ACS National |
| | 3-(3-pyridyl)-2-(E)- | | Meeting, Chicago, |
| | propenohydroxamic acid | | IL, Aug. 26-30, |
| | (Wakunaga Pharmaceutical Co.) | | 2001, MEDI-262. |
| T2 | N-hydroxy-2-[(4-methoxyphenyl)- | | WO 99/42436; |
| | sulphonyl]octanamide, (American | | Abstracts, 219th |
| | Cyanamid) | | ACS National |
| | | | Meeting, San |
| | | | Francisco, CA, |
| | | | Mar. 26-30, 2000, |
| | | | MEDI-281. |
| T3 | BB-1101, (2R,3S)—N4-hydroxy- | 147783-67-3 | U.S. Pat. No. 5,652,262 |
| | N1-[(1S)-2-(methylamino)-2-oxo- | | |
| | 1-(phenylmethyl)ethyl]-2-(2- | | |
| | methylpropyl)-3-(2-propenyl)- | | |
| | butanediamide | | |
| T4 | BB-1433, (2R,3S)—N1-[(1S)-1- | 147783-68-4 | WO 94/02447 |
| | (cyclohexylmethyl)-2-(methyl- | | |
| | amino)-2-oxoethyl]-N4,3- | | |
| | dihydroxy-2-(2-methylpropyl)- | | |
| | butanediamide | | |
| T5 | BB-94, batimastat, (2R,3S)—N4- | 130370-60-4 | WO 90/05719 |
| | hydroxy-N1-[(1S)-2-(methyl- | | |
| | amino)-2-oxo-1-(phenylmethyl)- | | |
| | ethyl]-2-(2-methylpropyl)-3-[(2- | | |
| | thienylthio)methyl]butanediamide | | |
| T6 | Ro-32-7315, (2R,3S,5E)-3- | 219613-02-2 | U.S. Pat. No. 6,235,787 |
| | [(hydroxyamino)carbonyl]-2-(2- | | |
| | methylpropyl)-6-phenyl-5-hexenoic | | |
| | acid, 2-(2-methylpropyl)-2- | | |
| | (methylsulphonyl)hydrazide (Roche) | | |
| T7 | GW-3333, (2R,3S)-3-(formyl- | 212609-68-2 | WO 98/38179 |
| | hydroxyamino)-4-methyl-2-(2- | | |
| | methylpropyl)-N-[(1S,2S)-2- | | |
| | methyl-1-[(2-pyridinylamino)- | | |
| | carbonyl]butyl]pentanamide | | |
| | (GlaxoSmithKline) | | |
| T8 | GW-4459, (2R,3S)-3-(formyl- | 260270-56-2 | WO 00/12466 |
| | hydroxyamino)-N-[(1S)-4-[[imino- | | |
| | (nitroamino)methyl] amino]-1-[(2- | | |
| | thiazolylamino)carbonyl]butyl]-2- | | |
| | (2-methylpropyl)hexanamide | | |
| | (GlaxoSmithKline) | | |
| T9 | GI 129471, (2R,3S)—N4-hydroxy- | 130370-59-1 | WO 90/05719 |
| | N1-[(1S)-2-(methylamino)-2-oxo- | | |
| | 1-(phenylmethyl)ethyl]-2-(2- | | |
| | methylpropyl)-3-[(phenylthio)- | | |
| | methyl]butanediamide (British | | |
| | BioTechnology) | | |
| T10 | CGS-33090A, (αR,1α,4β)-α-[[(4- | 209397-76-2 | U.S. Pat. No. 5,770,624 |
| | ethoxyphenyl)sulphonyl](4- | | |
| | pyridinylmethyl)amino]- | | |
| | N-hydroxy-4- | | |
| | propoxycyclohexaneacetamide | | |
| | (Novartis) | | |
| T11 | IK-682, 1-(αR,3S)-3-[4-[(3,5- | 223406-21-1 | U.S. Pat. No. 6,057,336 |
| | dimethylphenyl)methoxy]phenyl]- | | |
| | N-hydroxy-α,3-dimethyl-2-oxo- | | |
| | pyrrolidineacetamide (Bristol- | | |
| | Myers Squibb) | | |
| T12 | DPC-333, (αR)—N-hydroxy-α,3- | | U.S. Pat. No. 6,057,336 |
| | dimethyl-2-oxo-3-[4-(2-methyl-4- | | |
| | quinolinylmethoxy)phenyl]-1- | | |
| | pyrrolidineacetamide (Bristol- | | |
| | Myers Squibb) | | |
| T13 | TNF-484, (Novartis) | | |
| T14 | WTACE2, (Wyeth-Ayerst) | | |
| T15 | marimastat, (2S,3R)—N4-[(1S)-2,2- | 154039-60-8 | U.S. Pat. No. 5,986,132 |
| | dimethyl-1-[(methylamino)- | | |
| | carbonyl]propyl]-N1,2-dihydroxy- | | |
| | 3-(2-methylpropyl)butanediamide | | |
| | (British Biotechnology) | | |
| T16 | Ro 31-9790, (2R)—N1-[(1S)-2,2- | 145337-55-9 | U.S. Pat. No. 5,304,549 |
| | dimethyl-1-[(methylamino)- | | |
| | carbonyl]propyl]-N4-hydroxy-2-(2- | | |
| | methylpropy)butanediamide | | |
| | (Roche) | | |
| T17 | prinomastat, (3S)—N-hydroxy-2,2- | 192329-42-3 | WO 97/20824 |
| | dimethyl-4-[[4-(4-pyridinyloxy)- | | |
| | phenyl]sulphonyl]-3-thio- | | |
| | morpholinecarboxamide (Agouron) | | |
| T18 | (2S,3R)-2-cyclopentyl-N4-[(1S)- | 191613-76-0 | WO 97/19053 |
| | 2,2-dimethyl-1-[(methylamino)- | | |
| | carbonyl]propyl]-N1-hydroxy-3-(2- | | |
| | methylpropyl)butanediamide | | |
| | (British Biotechnology) | | |
| T19 | TAPI-O, N-[(2R)-2-[2-(hydroxy- | 163958-73-4 | WO 95/06031 |
| | amino)-2-oxoethyl]-4-methyl-1- | | |
| | oxopentyl]-3-(2-naphthalenyl)-L- | | |
| | alanyl-L-alaninamide (Immunex) | | |
| T20 | TAPI-1, N-[(2R)-2-[2-(hydroxy- | 163847-77-6 | U.S. Pat. No. 5,594,106 |
| | amino)-2-oxoethyl]-4-methyl-1- | | |
| | oxopentyl]-3-(2-naphthalenyl)-L- | | |
| | alanyl-N-(2-aminoethyl)-L- | | |
| | alaninamide (Immunex) | | |
| T21 | TAPI-2, N-[(2R)-2-[2-(hydroxy- | 187034-31-7 | U.S. Pat. No. 5,594,106 |
| | amino)-2-oxoethyl]-4-methyl-1- | | |
| | oxopentyl]-3-methyl-L-valyl-N-(2- | | |
| | aminoethyl)-L-alaninamide | | |
| | (Immunex) | | |
| T22 | CGS 27023A, (2R)—N-hydroxy-2- | 169799-04-6 | U.S. Pat. No. 5,455,258 |
| | [[(4-methoxyphenyl)sulphonyl](3- | | |
| | pyridinylmethyl)amino]-3-methyl- | | |
| | butanamide, monohydrochloride | | |
| | (Novartis) | | |
| T23 | [(5S)-5-[[(2R,3S)-2-(cyclohexyl- | 212609-63-7 | WO 98/38179 |
| | methyl)-3-(formylhydroxyamino)- | | |
| | 1-oxohexyl]amino]-6-oxo-6-(2- | | |
| | thiazolylamino)hexyl]carbamic | | |
| | acid, phenyl methyl ester (Glaxo) | | |
| T24 | CT-2256, (2S,3R)—N4-[(1S)-1- | 215593-63-8 | |
| | (aminocarbonyl)-2,2-dimethyl- | | |
| | propyl]-N1,2-dihydroxy-3-(2- | | |
| | methylpropryl)butanediamide | | |
| T25 | SP-057, (8S,11R,12S)—N12- | 191408-36-3 | WO 97/18207 |
| | hydroxy-11-(2-methylpropyl)-N8- | | |
| | [2-(4-morpholinyl)-2-oxoethyl]- | | |
| | 2,10-dioxo-1-oxa-3,9-diazacyclo- | | |
| | pentadecane-8,12-dicarboxamide | | |
| | (Dupont) | | |
| T26 | SL-422, (6S,7R,10S)—N6-hydroxy- | 191406-90-3 | WO 97/18207 |
| | N10-[2-(methylamino)-2-oxo- | | |
| | ethyl]-7-(2-methylpropyl)-8-oxo-2- | | |
| | oxa-9-azabicyclo[10.2.2]hexadeca- | | |
| | 12,14,15-triene-6,10-dicarboxamide | | |
| | (Dupont) | | |
| T27 | (8S,11R,12S)—N12-hydroxy-2,10- | 377088-88-5 | Xue, C.-B. et al., J. |
| | dioxo-N8-[2-oxo-2-(1-piperazinyl)- | | Med. Chem., 44(21), |
| | ethyl]-11-[[2′-(trifluoromethyl)- | | 3351-3354 (2001) |
| | [1,1′-biphenyl]-4-yl]methyl]-1-oxa- | | |
| | 3,9-diazacyclopentadecane-8,12- | | |
| | dicarboxamide (Dupont) | | |
| T28 | (8S,11R,12S)—N12-hydroxy-N8-[2- | 377088-85-2 | Xue, C.-B. et al., J. |
| | (4-morpholinyl)-2-oxoethyl]-2,10- | | Med. Chem., 44(21), |
| | dioxo-11-[[2′-(trifluoromethyl)- | | 3351-3354 (2001) |
| | [1,1′-biphenyl]-4-yl]methyl]-1-oxa- | | |
| | 3,9-diazacyclopentadecane-8,12- | | |
| | dicarboxamide (Dupont) | | |
| T29 | (3R)—N2-[(1,4-dihydro-4-oxo-8- | 204125-89-3 | WO 98/07742 |
| | quinazolinyl)sulphonyl]-N-hydroxy- | | |
| | 3-(2-methylpropyl)-L-a- | | |
| | asparaginyl-N,3-dimethyl-L- | | |
| | valinamide (AstraZeneca) | | |
| T30 | (2R,3S)—N1-(2,4-dioxo-1- | 277304-07-1 | WO 00/35885 |
| | imidazolidinyl)-N4-hydroxy-2-(2- | | |
| | methylpropyl)-3-[(2E)-3-phenyl-2- | | |
| | propenyl]butanediamide | | |
| | (Hoffmann-La Roche) | | |
| T31 | 5-bromo-N-hydroxy-2-[[(4- | 206547-73-1 | WO 98/16503 |
| | methoxyphenyl)sulphonyl](3- | | |
| | pyridinylmethyl)amino]-3- | | |
| | methylbenzamide (Wyeth-Ayerst) | | |
| T32 | FYK-1388, [2R-[1(S*),2R*,3S*]]- | 184947-94-2 | WO 96/33968 |
| | N1-[1-[[4-[(aminoiminomethyl)- | | |
| | amino]pheny]methyl]-2-(methyl- | | |
| | amino)-2-oxoethyl]-N4-hydroxy-2- | | |
| | (2-methylpropyl)-3-(3-phenyl- | | |
| | propyl)butanediamide, mono- | | |
| | acetate (salt) (Daiichi Seiyaku) | | |
| T33 | KB-R7785, (2S,3R)—N1-hydroxy- | 168158-16-5 | WO 95/04715 |
| | 2-methyl-N4-[(1S)-2-(methyl- | | |
| | amino)-2-oxo-1-phenylethyl]-3-(2- | | |
| | methylpropyl)butanediamide | | |
| | (Nippon Organon) | | |

The preferred TACE inhibitors include W-3646, Ro-32-7315, GW-3333, apratastat or (3S)—N-hydroxy-4[[4[(4-hydroxybut-2-ynyl)oxy]phenyl]sulfonyl]-2,2-dimethylthiomorpholine-3-carboxamide, GW-4459, CGS-33090A, DPC-333, TNF-484, WTACE2, SP-057, SL-422, FYK-1388 and KB-R7785.

The following TACE inhibitors are also advantageous: 3-[3-[N-isopropyl-N-(4-methoxyphenylsulfonyl)amino]phenyl]-3-(3-pyridyl)-2(E)-propenohydroxamic acid, (2R,3S)-3-(formylhydroxyamino)-4-methyl-2-(2-methylpropyl)-N-[(1S,2S)-2-methyl-1-[(2-pyridinylamino)carbonyl]butyl]pentanamide, (2R,3S)-3-(formylhydroxyamino)-N-[(1S)-4-[[imino(nitroamino)methyl]amino]-1-[(2-thiazolylamino)carbonyl]butyl]-2-(2-methylpropyl)hexanamide, (αR,1α,4β)-α-[[(4-ethoxyphenyl)sulfonyl](4-pyridinylmethyl)amino]-N-hydroxy-4-propoxycyclohexaneacetamide and (αR)—N-hydroxy-α,3-dimethyl-2-oxo-3-[4-(2-methyl-4-quinolinylmethoxy)phenyl]-1-pyrrolidineacetamide.

Table 3 below indicates the references of the patent applications describing various TACE inhibitors:

TABLE 3
TACE inhibitor reference:

| | | |
|---|---|---|
| EP 887077 | JP 11286455 | JP 11343279 |
| US 20010011134 | US 20010014688 | US 20010025047 |
| US 20010039287 | US 20010041710 | US 20010046989 |
| US 20010049449 | US 20010051614 | US 20010056088 |
| US 20020006922 | US 20020013333 | US 20020013341 |
| U.S. Pat. No. | U.S. Pat. No. 5,455,258 | U.S. Pat. No. 5,594,106 |
| 5,304,549 | | |
| U.S. Pat. No. | U.S. Pat. No. 5,652,262 | U.S. Pat. No. 5,665,777 |
| 5,629,285 | | |
| U.S. Pat. No. | U.S. Pat. No. 5,753,653 | U.S. Pat. No. 5,770,624 |
| 5,728,686 | | |
| U.S. Pat. No. | U.S. Pat. No. 5,817,822 | U.S. Pat. No. 5,872,146 |
| 5,776,961 | | |
| U.S. Pat. No. | U.S. Pat. No. 5,929,278 | U.S. Pat. No. 5,932,595 |
| 5,929,097 | | |
| U.S. Pat. No. | U.S. Pat. No. 5,955,435 | U.S. Pat. No. 5,962,481 |
| 5,952,320 | | |
| U.S. Pat. No. | U.S. Pat. No. 5,985,900 | U.S. Pat. No. 5,985,911 |
| 5,977,408 | | |
| U.S. Pat. No. | U.S. Pat. No. 6,013,649 | U.S. Pat. No. 6,057,336 |
| 5,986,132 | | |
| U.S. Pat. No. | U.S. Pat. No. 6,071,903 | U.S. Pat. No. 6,087,359 |
| 6,063,786 | | |
| U.S. Pat. No. | U.S. Pat. No. 6,100,266 | U.S. Pat. No. 6,114,372 |
| 6,090,840 | | |
| U.S. Pat. No. | U.S. Pat. No. 6,143,744 | U.S. Pat. No. 6,153,757 |
| 6,118,001 | | |
| U.S. Pat. No. | U.S. Pat. No. 6,172,064 | U.S. Pat. No. 6,180,611 |
| 6,172,057 | | |
| U.S. Pat. No. | U.S. Pat. No. 6,191,150 | U.S. Pat. No. 6,194,451 |
| 6,187,924 | | |
| U.S. Pat. No. | U.S. Pat. No. 6,197,795 | U.S. Pat. No. 6,200,996 |
| 6,197,791 | | |
| U.S. Pat. No. | U.S. Pat. No. 6,225,311 | U.S. Pat. No. 6,228,869 |
| 6,201,133 | | |
| U.S. Pat. No. | U.S. Pat. No. 6,235,787 | U.S. Pat. No. 6,268,379 |
| 6,235,730 | | |
| U.S. Pat. No. | U.S. Pat. No. 6,281,352 | U.S. Pat. No. 6,288,063 |
| 6,277,885 | | |
| U.S. Pat. No. | U.S. Pat. No. 6,313,123 | U.S. Pat. No. 6,326,516 |
| 6,310,088 | | |
| U.S. Pat. No. | U.S. Pat. No. 6,339,160 | U.S. Pat. No. 6,340,691 |
| 6,331,563 | | |
| U.S. Pat. No. | US H1992H1 | WO 0012466 |
| 6,342,508 | | |
| WO 0012467 | WO 0012478 | WO 0035885 |
| WO 0044709 | WO 0044710 | WO 0044711 |
| WO 0044713 | WO 0044716 | WO 0044723 |
| WO 0044730 | WO 0044740 | WO 0044749 |
| WO 0046189 | WO 0046221 | WO 0056704 |
| WO 0059285 | WO 0069812 | WO 0069819 |
| WO 0069821 | WO 0069822 | WO 0069827 |
| WO 0069839 | WO 0071514 | WO 0075108 |
| WO 0112592 | WO 0122952 | WO 0130360 |
| WO 0144189 | WO 0155112 | WO 0160820 |
| WO 0162733 | WO 0162742 | WO 0162750 |
| WO 0162751 | WO 0170673 | WO 0170734 |
| WO 0185680 | WO 0187870 | WO 0187883 |
| WO 0204416 | WO 0206215 | WO 9005719 |
| WO 9402447 | WO 9504715 | WO 9506031 |
| WO 9633166 | WO 9633167 | WO 9633968 |
| WO 9702239 | WO 9718188 | WO 9718207 |
| WO 9719050 | WO 9719053 | WO 9720824 |
| WO 9724117 | WO 9742168 | WO 9743249 |
| WO 9743250 | WO 9749674 | WO 9807742 |
| WO 9816503 | WO 9816506 | WO 9816514 |
| WO 9816520 | WO 9830541 | WO 9830551 |
| WO 9832748 | WO 9837877 | WO 9838163 |
| WO 9838179 | WO 9839326 | WO 9843963 |
| WO 9851665 | WO 9855449 | WO 9902510 |
| WO 9903878 | WO 9906410 | WO 9918076 |
| WO 9931052 | WO 9937625 | WO 9910080 |
| WO 9942436 | WO 9958531 | WO 9961412 |
| WO 9965867 | | |

The inhibitor compounds are formulated within a pharmaceutical composition, in combination with a pharmaceutically acceptable carrier. These compositions may be administered, for example, orally, enterally, parenterally, or topically. Preferably, the pharmaceutical composition is applied topically. When administered orally, the pharmaceutical composition may be in the form of tablets, gel capsules, dragees, syrups, suspensions, solutions, powders, granules, emulsions, or suspensions of microspheres or nanospheres or lipid or polymeric vesicles for controlled release. When administered parenterally, the pharmaceutical composition may be in the form of solutions or suspensions for infusion or for injection.

When applied topically, the pharmaceutical composition is more particularly for use in treating the skin and the mucous membranes, and may be in the form of salves, creams, milks, ointments, powders, impregnated pads, solutions, gels, sprays, lotions, suspensions or patches. It may also be in the form of suspensions of microspheres or nanospheres or of lipid or polymeric vesicles or of polymer patches or of hydrogels for controlled release. This composition for topical application may be in anhydrous form, in aqueous form or in the form of an emulsion. In a preferred variant, the pharmaceutical composition is in the form of a gel, a cream or a lotion.

The pharmaceutical composition may also contain inert additives or combinations of these additives, such as:

wetting agents;

flavor enhancers;

preservatives, such as para-hydroxybenzoic acid esters;

stabilizers;

moisture regulators;

pH regulators;

osmotic pressure modifiers;

emulsifiers;

UV-A and UV-B screening agents;

and antioxidants, such as alpha-tocopherol, butylhydroxyanisole or butylhydroxytoluene, superoxide dismutase, ubiquinol or certain metal-chelating agents.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method of screening for candidate compounds for the preventive and/or curative treatment of acne, comprising determining the ability of a compound to inhibit the expression or the activity of the TACE enzyme, or the expression of its gene or the activity of at least one of the promoters thereof, and thus identifying such candidate compound.

2. The in vitro method as defined by claim 1, comprising the following steps:
a. preparing at least two biological samples or reaction mixtures;
b. contacting one of the samples or reaction mixtures with one or more of the test compounds;
c. measuring the expression or the activity of the TACE enzyme, the expression of its gene or the activity of at least one of the promoters thereof, in the biological samples or reaction mixtures;
d. selecting the compounds for which an inhibition of the expression or of the activity of the TACE enzyme, or an inhibition of the expression of its gene or an inhibition of the activity of at least one of the promoters thereof, is measured in the sample or the mixture treated in b), compared with the untreated sample or mixture.

3. A regime or regimen for the preventive and/or curative treatment of acne, comprising administering to an individual in need of such treatment, a medicament which comprises a thus effective amount of at least one inhibitor of the TACE enzyme, said medicament being devoid of cyclooxygenase-2 inhibitor.

4. The regime or regimen as defined by claim 3, said at least one inhibitor of the TACE enzyme being selected from among E-3-[3-[N-(4-methoxyphenylsulfonyl)-N-isopropylamino]phenyl]-3-(3-pyridyl)-2(E)-propenohydroxamic acid, (2R,3S,5E)-3-[(hydroxyamino)carbonyl]-2-(2-methylpropyl)-6-phenyl-5-hexenoic acid 2-(2-methylpropyl)-2-(methylsulfonyl)hydrazide, (2R,3S)-3-(formylhydroxyamino)-4-methyl-2-(2-methylpropyl)-N-[(1S,2S)-2-methyl-1-[(2-pyridinylamino)carbonyl]butyl]pentanamide, apratastat, (2R,3S)-3-(formylhydroxyamino)-N-[(1S)-4-[[imino(nitroamino)methyl]amino]-1-[(2-thiazolylamino)carbonyl]butyl]-2-(2-methylpropyl)hexanamide, (αR,1α,4 β)-α-[[(4-ethoxyphenyl)sulfonyl](4-pyridinylmethyl)amino]-N-hydroxy-4-propoxycyclohexaneacetamide, (αR)-N-hydroxy-α,3-dimethyl-2-oxo-3-[4-(2-methyl-4-quinolinylmethoxy)phenyl]-1-pyrrolidineacetamide, TNF-484, WTACE2, (8S,11R,12S)-N12-hydroxy-11-(2-methylpropyl)-N8-[2-(4-morpholinyl)-2-oxoethyl]-2,10-dioxo-1-oxa-3,9-diazacyclopentadecane-8,12-dicarboxamide, (6S,7R,10S)-N-6-hydroxy-N10-[2-(methylamino)-2-oxoethyl]-7-(2-methylpropyl)-8-oxo-2-oxa-9-azabicyclo[10.2.2]hexadeca-12,14,15-triene-6,10-dicarboxamide, [2R-[1(S*),2R*,3S*]]-N-1-[1-[[4-(aminoiminomethyl)amino]phenyl]methyl]-2-(methylamino)-2-oxoethyl]-N4-hydroxy-2-(2-methylpropyl)-3-(3-phenylpropyl)butanediamide monoacetate, (2S,3R)-N1-hydroxy-2-methyl-N-4-[(1S)-2-(methylamino)-2-oxo-1-phenylethyl]-3-(2-methylpropyl)butanediamide, 3-[3-[N-isopropyl-N-(4-methoxyphenylsulfonyl)amino]phenyl]-3-(3-pyridyl)-2(E)-propenohydroxamic acid, (2R,3S)-3-(formylhydroxyamino)-4-methyl-2-(2-methylpropyl)-N-[(1S,2S)-2-methyl-1-[(2-pyridinylamino)carbonyl]butyl]pentanamide, (2R,3S)-3-(formylhydroxyamino)-N-[(1S)-4-[[imino(nitroamino)methyl]amino]-1-[(2-thiazolylamino)carbonyl]butyl]-2-(2-methylpropyl)hexanamide, (αR,1α,4β)-α-[[(4-ethoxyphenyl)sulfonyl](4-pyridinylmethyl)amino]-N-hydroxy-4-propoxycyclohexaneacetamide, and (αR)-N-hydroxy-α3-dimethyl-2-oxo-3-[4-(2-methyl-4-quinolinylmethoxy)phenyl]-1-pyrrolidineacetamide.

5. The regime or regimen as defined by claim 3, said at least one TACE-enzyme inhibitor comprising the only active ingredient(s) in said medicament.

6. The regime or regimen for the prevention and/or curative treatment of acne as defined by claim 3, said at least one TACE-enzyme inhibitor comprising a compound identified by determining the ability of such compound to inhibit the expression or the activity of the TACE enzyme, or the expression of its gene or the activity of at least one of the promoters thereof.

7. The regime or regimen as defined by claim 3, said medicament being formulated for topical application.
